(19) United States
(12) Patent Application Publication
(10) Pub. No.: US 2024/0374790 A1
(43) Pub. Date: Nov. 14, 2024

(54) Rollable Biological Construct

(71) Applicant: Cannuflow, Inc., Scotts Valley, CA (US)

(72) Inventor: Theodore R. Kucklick, Scotts Valley, CA (US)

(73) Assignee: Cannuflow, Inc., Scotts Valley, CA (US)

(21) Appl. No.: 18/658,398

(22) Filed: May 8, 2024

Related U.S. Application Data
(60) Provisional application No. 63/501,545, filed on May 11, 2023.

(51) Int. Cl. A61L 27/24; A61L 27/56

(52) U.S. Cl. CPC A61L 27/24; A61L 27/56; A61L 2430/10

(57) ABSTRACT

A biologic construct having a resilient polymer layer or substrate on a first surface and a porous collagen sheet or layer on a second surface of the rollable construct. The resilient polymer layer and the porous collagen sheet or layer can be laminated together or can optionally include a bladder between the resilient polymer layer and the collagen sheet to form a reservoir between the resilient polymer layer and the collagen layer. The biologic construct is rollable in a first constrained configuration and a second unfurled configuration.

### RELATED PATENT APPLICATION

This application claims priority to U.S. Provisional Application 63/501,545 filed May 11, 2023.

### FIELD OF THE INVENTIONS

The present application relates to treatment of torn tendon tissue, in particular to a device and method of delivering a rollable implant to a torn tendon.

### BACKGROUND OF THE INVENTIONS

Regenerative tissue engineering is a dynamic field that has proposed solutions to achieving functional tissue regeneration. Regenerative tissue or materials include cells, scaffolds or hydrogels and growth stimulating factors. As soon as the cells are in contact with a scaffold, biology differentiates into materiobiology, and the behavior of the cells depend on the scaffold microstructure, surface properties, and mechanical properties.

Delivery of biological constructs to a torn tissue site present challenges to ensure the biological construct is delivered most efficiently without comprising structure of the biological construct while being delivered to the desired treatment site.

### SUMMARY

The devices and methods described below provide for an improved regenerative biological construct or patch that is rollable for improved delivery to a preferred surgical site. The biological construct can be efficiently delivered and placed within the patient to adjust for functionality of delivery and ultimate placement and securing to the patient.

A rollable regenerative construct includes a resilient polymer layer or substrate on a first surface and a porous collagen sheet or layer on a second surface of the rollable construct. The resilient polymer layer and the porous collagen sheet or layer can be laminated together. The rollable regenerative biological construct can also include an elongated or spherical shaped bladder between the resilient polymer layer and the collagen sheet to form a reservoir between the resilient polymer layer and the collagen layer.

### BRIEF DESCRIPTION OF THE DRAWINGS

FIG. 1 illustrates a first view of a rollable regenerative biological construct in an unfurled or expanded configuration.

FIG. 2 illustrates an opposite view of the biological construct in an unfurled configuration.

FIG. 3 illustrates an exploded view of the individual layers of an alternative biological construct.

FIG. 4 illustrates a biological construct in a furled configuration.

FIG. 5 illustrates a biological construct in a furled configuration inserted within a delivery tube.

FIG. 6 illustrates the test method for determining the unfurled and furled configurations of the biological construct.

FIGS. 7a, 7b, 8 and 9 illustrate exploded views of alternative rollable biological constructs.

FIG. 10 illustrates an alternative rollable biological construct with a spring element on the first layer.

FIG. 11 illustrates a funnel delivery tube and cannula for inserting a rolled regenerative biological construct.

### DETAILED DESCRIPTION OF THE INVENTIONS

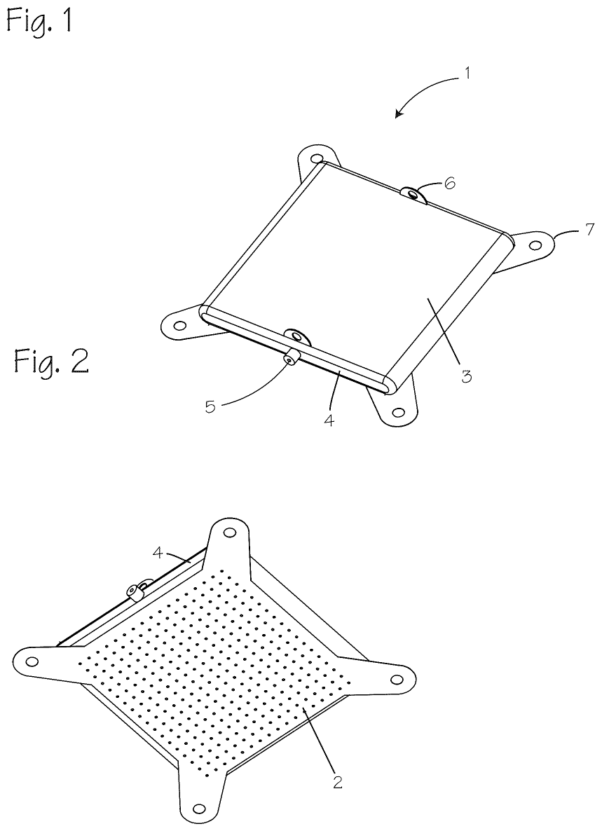

FIG. 1 illustrates a first view of a rollable regenerative biological construct 1 in an unfurled or expanded configuration and FIG. 2 illustrates an opposite view of the biological construct. The rollable construct includes a resilient polymer layer or substrate 2 on one surface of the biological construct and a porous collagen sheet 3 or layer on the opposite side of the rollable construct. The resilient polymer layer serves as a scaffold or structural sheet of the biological construct. It can include a plurality of interconnected structural fibers. The resilient polymer layer and the porous collagen sheet or layer can be laminated together to form the biological construct. Alternatively, the rollable regenerative biological construct can include an elongated or spherical shaped bladder 4 between the resilient polymer layer and the collagen sheet to serve as a reservoir for fluid such as blood, platelet rich plasma (PRP), stem cells, or other fluid or other fluent medication such as an anti-inflammatory or antibiotic. The biological construct may optionally include a fluid port 5 at a first end of the bladder and a delivery tab 6 at a second end of the bladder for releasable attachment of the biological construct to a delivery device for placement within the body. The biological construct may also include fixation tabs or grommets 7 for securing the biological construct to a desired tissue location within the body.

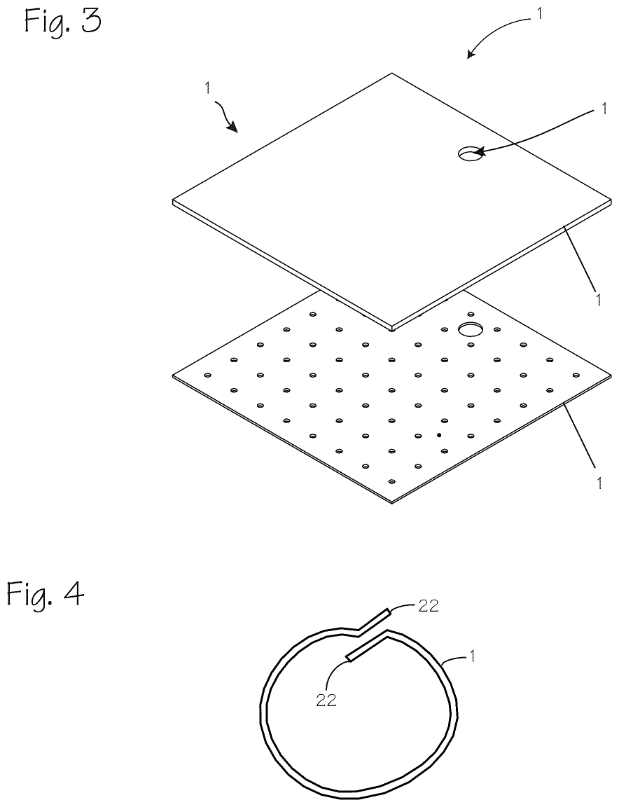

FIG. 3 illustrates an exploded view of the individual layers of another biological construct. This Figure illustrates the resilient polymer layer 2 or substrate with a porous collagen sheet 3 on top of the resilient polymer layer but does not include an intermediate bladder between the layers. The layers are laminated together. The resilient polymer layer and the porous collagen layer may include an engagement tab 8 for engagement of a driver for introduction into a patient. This figure illustrates the biological construct in an unfurled configuration. FIG. 4 illustrates the biological construct in a furled configuration. This figure illustrates the edges of the biological construct in the furled configuration can include a fold or chamfer 22 on each of the edges of the construct. The fold or chamfer allows one edge to fold under the other edge when in a furled configuration. This provides the benefit that the folded under edge does not hit and interfere with furling the construct.

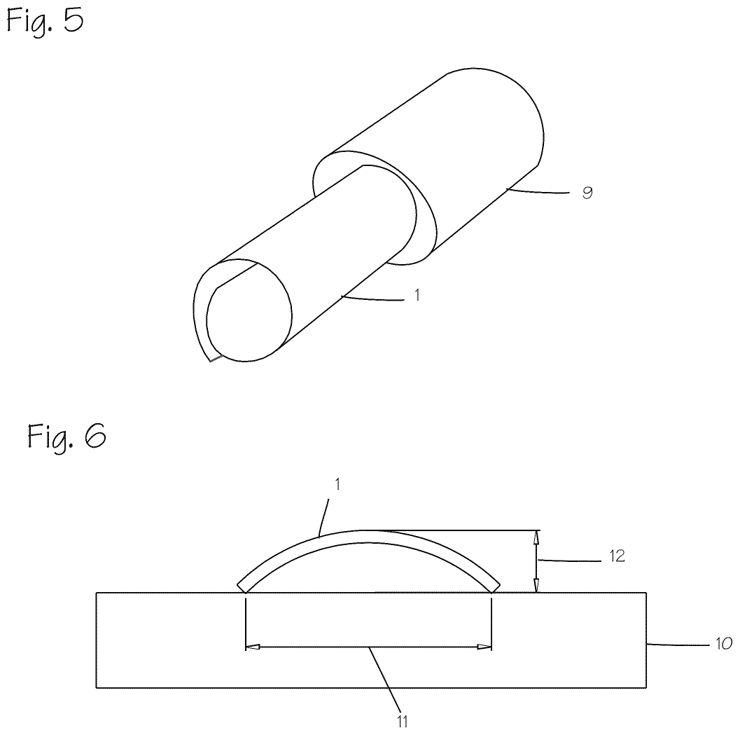

FIG. 5 illustrates a biological construct in a furled configuration inserted within a delivery tube 9. FIG. 6 illustrates the test method for determining the unfurled and furled configurations of the biological construct. In a first configuration, the construct is inserted into the delivery tube and constrained within the delivery tube for at least one minute. The unfurled implant is placed on a surface plate 10 and has a width 11 and a height 12. The constrained sheet is deformed into a half circle having a height to width ratio for a half-circle of ½ or 0.5. In the first configuration, a height to width ratio of ½ is the maximum acceptable deformation. After the construct is constrained for at least a minute, the unfurled configuration is placed on the surface plate and the strain recovery ratio is determined, also as a ratio of height to width. If there is 100% strain recovery the construct is expanded into a flat configuration. Furling and unfurling defines the movement of biological material from a compact or rolled configuration to an open or unconstrained configuration. In a furled configuration, the biological construct is wound or rolled up to fit within the delivery tube for efficient placement within the delivery device for advancement to desired treatment location. Once delivered to desired site and unfurled, the biological construct gradually expands or unfolds to desired size and shape to conform to tissue in order to be attached most efficiently. The strain recovery of the second configuration recovers to a full elastic recovery, or to a flat configuration. This height to width ratio is 0, where the height is 0 (full elastic recovery) and the width is the predetermined width of the construct. In a second configuration, the construct maximum is full elastic recovery, or 0. Thus the range of the height to width ratio in a furled configuration to an unfurled configuration is from ½ (half circle) to 0 (flat), which defines the range of conditions between full elastic recovery and the maximum acceptable deformation.

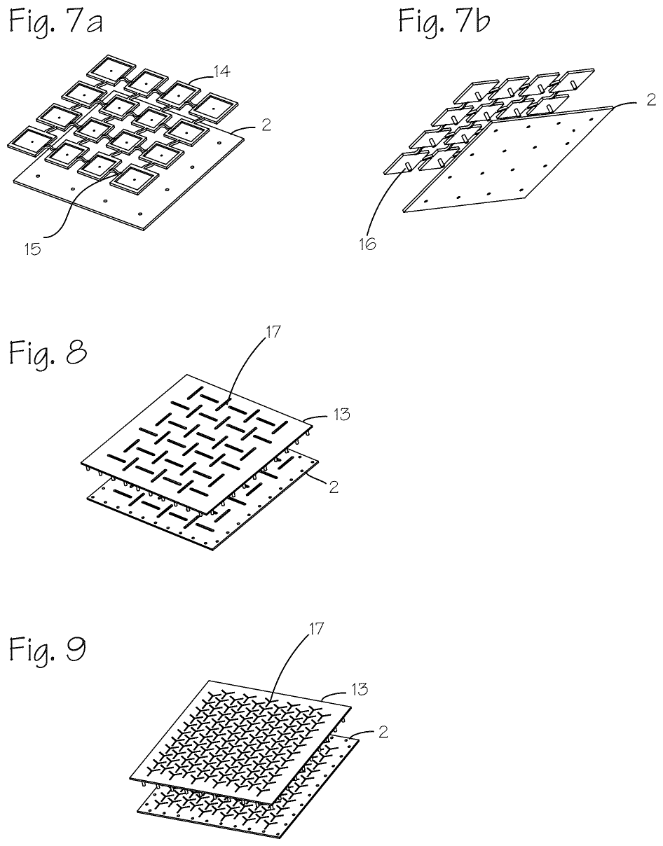

FIGS. 7a, 7b, 8 and 9 illustrate exploded views of alternative rollable biological constructs in unfurled configurations. FIGS. 7a and 7b illustrate a resilient polymer layer or substrate 2 with a second layer 13 that includes a plurality of compartmentalized cells 14. The cells can be of varying sizes and shapes. FIG. 7a illustrates a plurality of square cells interconnected via flexible springs 15 between each cell. Each cell can accommodate desired fluids or growth promotion materials to improve recovery time when placed over damages tissue. The second layer includes pins 16 that project from one side of the second layer to connect the first resilient polymer layer to the second layer. The pins also serve as an array to direct fluid from the compartmentalized cells onto damaged tissue. FIGS. 8 and 9 illustrate biological constructs that include a first layer or sheet and a second layer or sheet overlaying the first layer. Each of the layers are formed of auxetic kirigami metamaterials and include various slit patterns. The material is made up of repeating cuts and folds 17. The patterns allow the material to exhibit unique mechanical properties such as the ability to expand and contract in specific ways when subjected to bending forces. In addition, the metamaterial is lightweight and flexible and the thin sheets can be stacked together to create a biological construct that is lightweight and flexible yet exhibits great strength. The alternative biological constructs of FIGS. 7a, 7b, 8 and 9 have a height and a width and can be furled into a second constrained position where the recovery strain of the second configuration recovers to a full elastic recovery, or to a flat configuration. This height to width ratio is 0, where the height is 0 (full elastic recovery) and the width is the predetermined width of the construct. In a second configuration, the construct maximum is full elastic recovery, or 0. Thus the range of the height to width ratio in a furled configuration to an unfurled is from ½ (half circle) to 0 (flat), which defines the range of conditions between full elastic recovery and the maximum acceptable deformation.

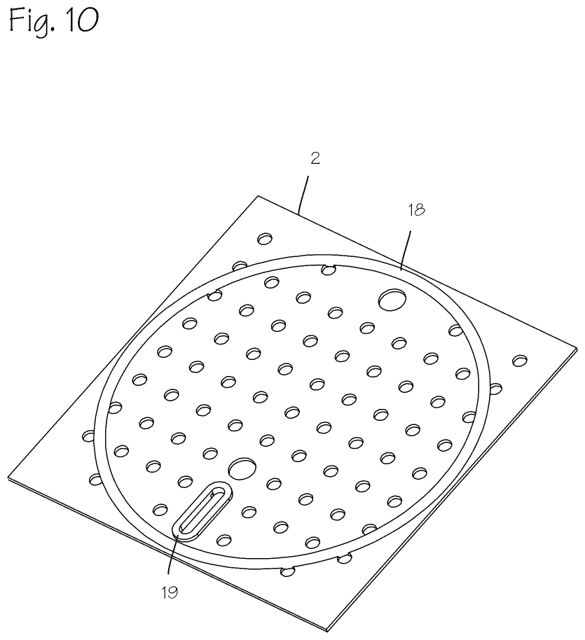

FIG. 10 illustrates an alternative rollable biological construct with a spring element 18 in an unfurled configuration. The biological construct includes a first layer and second layer. The first layer includes a ring-shaped spring 18 and an opening 19 to engage a delivery device. The ring shape of the spring provides foldability to the biologic construct. The spring element can be integrally printed or molded onto the first layer. The rollable biological construct is then coated with collagen or alternatively the collagen may be printed into the construct to form the collagen sheet over the resilient polymer layer. The sheet is a microporous scaffold construction with a pore size of between 10-600 μm.

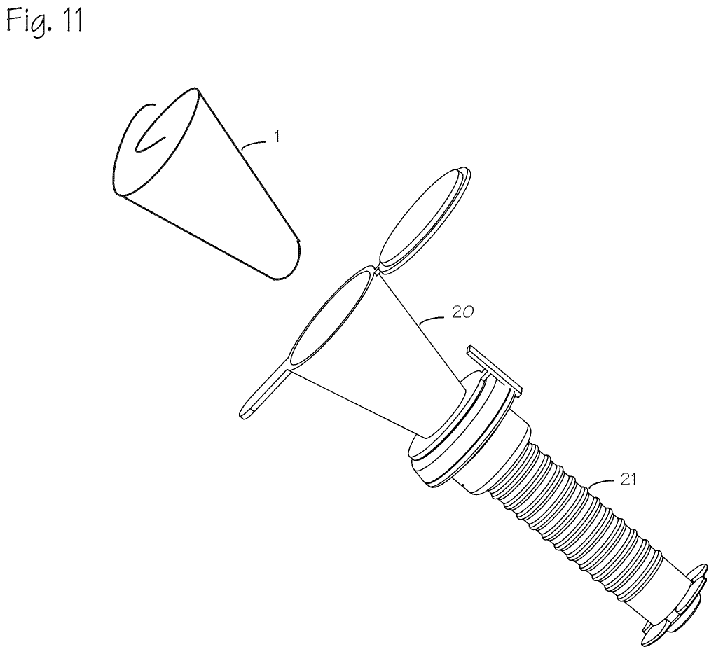

FIG. 11 illustrates a funnel delivery tube 20 and cannula 21 for inserting a rolled regenerative biological construct 1. In use, the funneled delivery tube is connected to an end of the cannula. When the funnel is connected, the rolled regenerative biological construct is introduced into the funnel. The construct is introduced in an initial furled configuration. The construct is inserted into the funneled delivery tube, which has an inner diameter between 3.5 mm and 10 mm and held in a rolled and constrained condition. Constraining for at least one minute configures the biological construct to the desired springiness for delivery through the cannula. The construct is then pushed out via a delivery device to a desired treatment site and allowed to unfurl or expand. The delivery device pushes the construct into position from the front or distal edge. Once delivered and in an unfurled or expanded configuration the strain recovery of the construct can be measured on a surface plate in a test for deformation. The unfurled implant is placed on a surface plate that has a width and a height. The constrained sheet is deformed into a half circle having a height to width ratio for a half-circle of ½ or 0.5. In the first configuration, a height to width ratio of ½ is the maximum acceptable deformation. After the construct is constrained for at least a minute, the unfurled configuration is placed on the surface plate and the strain recovery ratio is determined as a ratio of height to width. If there is 100% strain recovery the construct is expanded into a flat configuration. After determining the unfurled configuration is within the desired range, then it is secured into target tissue and the delivery device is removed.

The resilient polymer layer can include a drug coating on a surface of the polymer layer that is facing the porous collagen layer. Drug coating may include small molecule drugs such as metformin, TIMPS (tissue inhibitors of metalloproteinases) hydroxyapatite, hyaluronic acid, or biologics such as Bone Morphogenic Proteins (BMP's), growth factors such as TGF or EGF or other regenerative agents that are capable of being coated on to a polymer layer. The resilient polymer layer can be perforated, or die or laser cut. The resilient polymer layer can be made of polycaprolactone family material or any similar polymer. The resilient polymer is between 0.1 mm and 1 mm thick. The resilient polymer layer or substrate can encapsulate fluid to deliver it to a predetermined location of the body over time. It provides structural support but also allows for flexibility and movement and enhances the durability and longevity of the biologic construct.

The resilient polymer sheet or substrate can be made of any bioabsorbable polymer such as Polyglycolic Acid, Poly-Lactic Acid, Poly-L-Lactic Acid (PGA, PLLA), Polyglycolic Acid (PGA), Poliglecaprolactone (PLCL), Polycaprolactone (PCL), Polydioxanone (PDS), Polyglyconate, or copolymers such as poly-L-lactide-eco-ε-caprolactone (PLACL). The use of L-lactide-eco-ε-caprolactone provides the advantage of quick degradation and spring and shape memory qualities which provide for proper placement at a preferred surgical site. The resilient polymer sheet can also be made of a fiber reinforced material such as PLGA fibers or other absorbable fiber or suture material or can alternatively be reinforced with a nanomaterial such as carbon nanotubes or other non-porous material. The porous collagen layer can be made of any open cell construction. The pore size can range from about 10 microns to 600 microns. The porous collagen layer thickness may be about 0.2 mm to 5 mm thick. The entire biological construct can be square, rectangular or any other shape. For example, each side ranging in area from about 100 mm to 2500 mm square. The entire biological construct can optionally be coated in collagen. The porous layer includes porous structure that allows for cell attachment and integration of the biological construct with tissue.

The collagen sheet or layer can be made of any material or polymer derived from natural resources such as plants, animals or microorganisms. Specifically, the materials can be derived from any animal, such as bovine or porcine or human donor. The material could also be non-animal derived or plant based recombinant collagen, for example Evonik Vecollan.

The micropore features of the construct may be molded with pore forming removable material, foamed, made from a fibrous construct, 3D printed, or 3D printed with a removable material such as PVA, or may be laser drilled. The laser may be motion controlled, or projected through a pattern mask. The pore sizes can be between 10-600 μm and can be sized and shaped to be suitable for a tissue scaffold, or elution of a growth promoting substance such as PRP (platelet rich plasma) or a medication, or combination thereof.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A rollable biological construct comprising:
a resilient biocompatible polymer substrate having a height and a width; and
a porous collagen layer formed over the resilient biocompatible polymer substrate;
wherein the rollable biological construct has a first rolled configuration that is furled and a second configuration that is unfurled, in the first furled configuration the resilient biocompatible layer of the construct is rolled into a half circle having a height to width ratio of ½ or less and in the second or unfurled configuration the height to width ratio is at most 0 percent of the first furled configuration.

2. The rolled biological construct of claim 1 further comprising a bladder between the polymer substrate and the porous collagen layer.

3. The rolled biological construct of claim 1 further comprising a chamfer at each edge of the biological construct.

4. The rolled biological construct of claim 2 further comprising a fluid port at a first end of the bladder.

5. The rolled biological construct of claim 3 further comprising a delivery tab at a second end of the bladder.

6. The rolled biological construct of claim 4 further comprising at least one fixation tab or grommet.

7. A biological construct system comprising:
a catheter having a delivery tube with an inner diameter of between 3.5 mm and 10 mm; and
a biological construct having a resilient biocompatible polymer substrate having a height and a width and a porous collagen layer formed over the resilient biocompatible polymer substrate, wherein the biological construct has a first rolled configuration that is furled and a second configuration that is unfurled, in the first furled configuration the resilient biocompatible layer of the construct is rolled into a half circle having a height to width ratio of ½ or less and in the second or unfurled configuration the height to width ratio is at most 0 percent of the first furled configuration.

8. The biological construct system of claim 7 wherein the biological construct further comprises a bladder between the polymer substrate and the porous collagen layer.

9. A method of inserting rolled biological construct to a predetermined tissue location comprising:
introducing the rolled biological construct having a height and a width into a delivery tube in an initial furled configuration and constraining the biological construct within the delivery tube, where the initial furled configuration of the resilient biocompatible layer of the construct is rolled into a half circle having a height to width ratio of ½ or less;
advancing the biological construct via a delivery device to a desired treatment site and allowing the biological construct to unfurl or expand to a second configuration, where in the unfurled configuration the height to width ratio is at most 0 percent of the first furled configuration;
placing the biologic construct onto the predetermined tissue in the unfurled configuration and securing the biological construct to the tissue; and
removing the delivery device.
